(19) United States
(12) Patent Application Publication
(10) Pub. No.: US 2010/0204614 A1
(43) Pub. Date: Aug. 12, 2010

(54) ELECTRONIC SNORE RECORDING DEVICE AND ASSOCIATED METHODS

(75) Inventors: Sherrill F. Lindquist, Melbourne, FL (US); John E. Zurasky, Merritt Island, FL (US); Jacob D. Zurasky, Merritt Island, FL (US)

Correspondence Address: ALLEN, DYER, DOPPELT, MILBRATH & GILCHRIST P.A., 1401 CITRUS CENTER 255 SOUTH ORANGE AVENUE, P.O. BOX 3791, ORLANDO, FL, 32802-3791, US

(73) Assignee: ZURLIN TECHNOLOGIES HOLDINGS, LLC, Melbourne, FL (US)

(21) Appl. No.: 12/699,200

(22) Filed: Feb. 3, 2010

Related U.S. Application Data
(63) Continuation-in-part of application No. 12/154,339, filed on May 22, 2008.
(60) Provisional application No. 60/946,159, filed on Jun. 26, 2007.

(51) Int. Cl. A61B 7/00

(52) U.S. Cl. 600/586

(57) ABSTRACT

The snore recording device includes a portable housing, a microphone carried by the housing for capturing an audio input signal including snoring, a memory, such as a removable memory, carried by the housing, and processing circuitry carried by the housing and coupled to the microphone and the memory. The processing circuitry is for low pass filtering the audio input signal from the microphone to generate a low pass filtered analog signal, performing analog-to-digital conversion (ADC) on the low pass filtered analog signal to generate an intermediate digital signal, performing a moving average filtering of the intermediate digital signal to generate moving average intensity data, performing a Fast Fourier Transform (FFT) on the intermediate digital signal to generate frequency component data, and storing at least the moving average intensity data and frequency component data in the memory.

### CROSS-REFERENCE TO RELATED APPLICATIONS

The present application is a CIP of U.S. Utility application Ser. No. 12/154,339 filed May 22, 2008 and which claims priority from U.S. Provisional Application No. 60/946,159, filed Jun. 26, 2007, entitled “Electronic Anti-Snoring & Sleep Apnea Device (EAS/SAD) For Sleep-Breathing Disorders, Electronic Anti-Bruxing Device, And Electronic Device For TMD Therapy” by Lindquist et al., which are hereby incorporated by reference in its entirety.

### COPYRIGHT NOTICE

A portion of the disclosure of this patent document contains material which is subject to copyright protection. The copyright owner has no objection to the facsimile reproduction by any one of the patent document or the patent disclosure, as it appears in the Patent and Trademark Office patent file or records, but otherwise reserves all copyright rights whatsoever.

### BACKGROUND OF THE INVENTION

1. Field of the Invention

The invention is directed to devices and methods for analyzing sleep-disordered breathing, and, more particularly, to electronic devices for monitoring snoring and processing recorded audio data.

2. Description of the Prior Art

Current treatments for snoring and Obstructive Sleep Apnea (OSA) include behavioral changes such as losing weight, avoiding alcohol, tobacco, sleeping pills, and attempting to adjust sleeping position. Continuous Positive Airway Pressure (CPAP) can be effective but very uncomfortable and noisy to wear during the night with only 50% patient compliance. Oral appliance therapy is available but many times can cause facial pain, TMD symptoms, and changes in tooth position and occlusion. Surgical approaches are available but most are quite drastic requiring patients to undergo unwanted procedures.

An example of one approach is presented in U.S. Pat. No. 5,792,067 to Karell which is directed to a device and method for addressing sleep and other disorders through electromuscular stimulation within specific areas of a patient's mouth. A mouthpiece includes an electrode for stimulating either the hard palate, soft palate or the pharynx. The mouthpiece includes a denture-like plate to which the control unit and electrodes may be attached.

Also, snoring is an extremely common condition and it has been estimated that up to 50% of the adult population snores. According to the National Sleep Foundation, 90 million Americans suffer from snoring or obstructive sleep apnea. A snore is a respiratory noise generated by turbulent air flowing through an occluded airway during sleep causing vibration of the tissues in the oropharynx. Decreased levels of airway muscle tone is the key factor. Snoring, gasping for air, and cessation of breathing are possible symptoms of obstructive sleep apnea (OSA).

During sleep, the OSA sufferer cycles through a series of events: The airway becomes blocked, the patient gets no air; blood oxygenation saturation (SaO₂) decreases, causing the heart to pump faster; momentary sleep arousal occurs to restore breathing; disturbed sleep is recycled until the next apnea, possibly hundreds of times per night.

Snoring has been identified by observation, patient history, and can be estimated on the polysomnogram (PSG). No accurate and consistent system of recording and scoring of snoring has been available. The polysomnograph developed by Dr. Nathaniel Kleitman in the 1950s, records multiple physiological activities during sleep including: Electroencephalogram EEG (brain electrical activity); Electroculogram EOG (eye movement); Electromyogram EMG (jaw muscle movement); Leg muscle movement; Airflow; Respiratory effort (chest and abdominal excursion); Electrocardiogram ECG; Oxygen saturation SaO₂; and Audio and visual recording of nocturnal sounds and movements.

Snoring analysis is important for the diagnosis and treatment of sleep-related breathing disorders but has traditionally been assessed in clinical practice from subjective accounts by the snorer and his/her partner. The use of polysomnographic recording is the standard evaluation procedure. The present graphic representation of the snoring sounds on the PSG is not definitive and there is a need for enhancement of quality and quantification.

### SUMMARY OF THE INVENTION

Objects, advantages and features in accordance with the present invention are provided by a snore recording device including a portable housing, a microphone carried by the housing for capturing an audio input signal including snoring, a memory, such as a removable memory, carried by the housing, and processing circuitry carried by the housing and coupled to the microphone and the memory. The processing circuitry is for low pass filtering the audio input signal from the microphone to generate a low pass filtered analog signal, performing analog-to-digital conversion (ADC) on the low pass filtered analog signal to generate an intermediate digital signal, performing a moving average filtering of the intermediate digital signal to generate moving average intensity data, performing a Fast Fourier Transform (FFT) on the intermediate digital signal to generate frequency component data, and storing at least the moving average intensity data and frequency component data in the memory.

The processing circuitry may also be for calculating, from the moving average intensity data, snoring index data based upon a number of snoring events per unit time, and storing the snoring index data in the memory. The processing circuitry may also be for amplifying the audio input signal from the microphone. The processing circuitry may also be for storing the low pass filtered analog signal in the memory. The processing circuitry may further comprise a polysomnograph (PSG) interface for interfacing to a PSG. The processing circuitry may also be for performing a circular buffering of the intermediate digital signal.

A method aspect of the invention is for recording snores and includes capturing an audio input signal including snoring, low pass filtering the audio input signal to generate a low pass filtered analog signal, performing analog-to-digital conversion (ADC) on the low pass filtered analog signal to generate an intermediate digital signal, performing a moving average filtering of the intermediate digital signal to generate moving average intensity data, performing a Fast Fourier Transform (FFT) on the intermediate digital signal to generate frequency component data, and storing at least the moving average intensity data and frequency component data in a memory.

### BRIEF DESCRIPTION OF THE DRAWINGS

FIG. 1 is a drawing of a maxillary stone cast with a thin plastic sheet adapted to it used to fabricate the maxillary plastic arch form for the electronic components of the intra-oral appliance in accordance with the present invention.

FIG. 2 is a drawing of the rechargeable battery and electronic transceiver located in the palatal aspect of the intra-oral appliance. Also displayed are the circuit extension leads and contacts which stimulate the hamular notches.

FIG. 3A is a bottom view of the intra-oral appliance including the electronics being sandwiched between thin protective layers.

FIG. 3B is a cross-sectional view of the intra-oral appliance taken along the line B-B of FIG. 3A.

FIG. 4 is a drawing of the extra-oral unit housing the microphone, signal processor, battery charger, and the data recorder which is placed on the patient's nightstand.

FIGS. 5A and 5B are simplified charts of the electronic functions of a first version of the remote unit and intra-oral appliance, respectively.

FIG. 6 is a drawing of the intra-oral appliance for bruxism showing bruxism detection sensors in the form of a pressure sensitive electro conductive rubber sensor or pressure receptor switch and the electrical stimulation points.

FIG. 7 is a drawing of the intra-oral appliance for TMD showing design with pressure sensitive electro conductive rubber sensors or pressure receptor switches to detect occlusal para-function and the electrical stimulation points.

FIG. 8 is a high-level block diagram of the hardware architecture of a mouthpiece unit in accordance with one aspect of the invention.

FIG. 9 is a high-level block diagram of the hardware architecture of a nightstand unit in accordance with one aspect of the invention.

FIGS. 10-12 are more detailed schematic diagrams of the hardware architecture of the mouthpiece unit and nightstand unit of FIGS. 8 and 9.

FIG. 13 is a high-level block diagram of the software architecture implemented in firmware for the mouthpiece unit in accordance with one aspect of the invention.

FIG. 14 is a high-level block diagram of the software architecture implemented in firmware for the nightstand unit in accordance with one aspect of the invention.

FIG. 15 is a block diagram illustrating an embodiment of the extra-oral unit of FIG. 4 being used as a snore recording device.

FIG. 16 is a block diagram illustrating an embodiment of the firmware architecture of the snore recording device of FIG. 15.

### DETAILED DESCRIPTION OF THE INVENTION

The present invention will now be described more fully hereinafter with reference to the accompanying drawings in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout. The dimensions of layers and regions may be exaggerated in the figures for greater clarity.

FIG. 1 is an illustration of a snoring and OSA patient's maxillary teeth. The cast 100 is fabricated by the dentist or dental assistant making alginate (irreversible hydrocolloid) impressions of the maxillary and mandibular arches in the usual way impressions are made. A vacuum thermoforming machine (such as manufactured by Raintree Essix Inc., Metairie, La.) can be used to pull down sufficiently heated plastic onto the maxillary model, as would be appreciated by those skilled in the art. This plastic material 102 will become the arch form base upon which a rechargeable battery and the electronic transceiving unit will be mounted. After these components are mounted in the palatal aspect of the arch, a second “sandwiching” piece of thin plastic is vacuum formed over the electronic components to protect them from saliva.

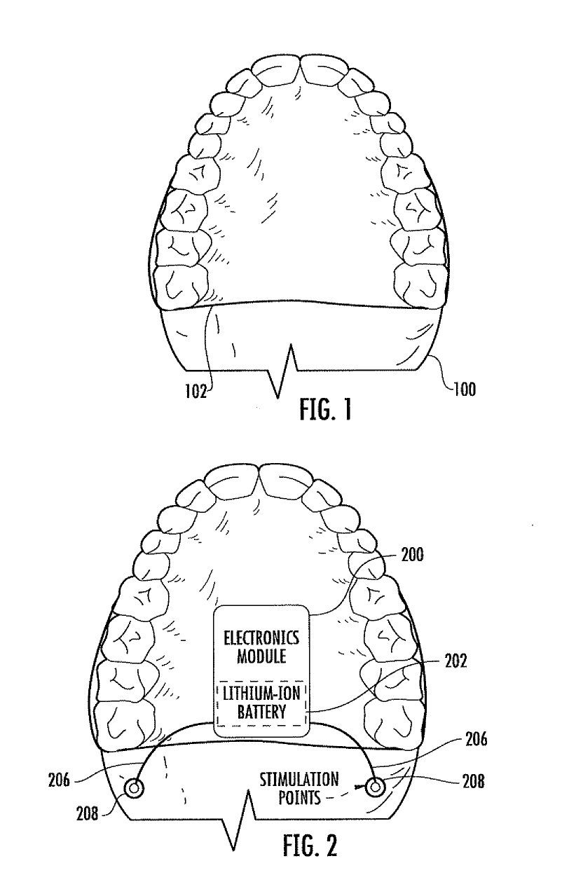

FIG. 2 is an illustration of the electronics module or transceiving unit 200 including rechargeable battery 202, and circuit extension leads 206 and associated tissues contacts 208 which contact the hammular notches bilaterally. The battery 202 used in the unit must be of sufficient voltage in order to create the necessary tone in the musculature involved with soft palate flexing or stiffening (tensor veli palatini muscles and the levator veli palatini muscles). When not in use, the intra-oral member should be recharged during the day. Wire leads 206 from the electronic circuit are preferably 28 gauge wire and run between the “sandwiched” plastic arch form distal to the maxillary 2nd molars and terminate with the circuit extension contacts 208, such as stainless orthodontic ballclamps (0.28 in (0.7 mm)) which contact in the hamular notch.

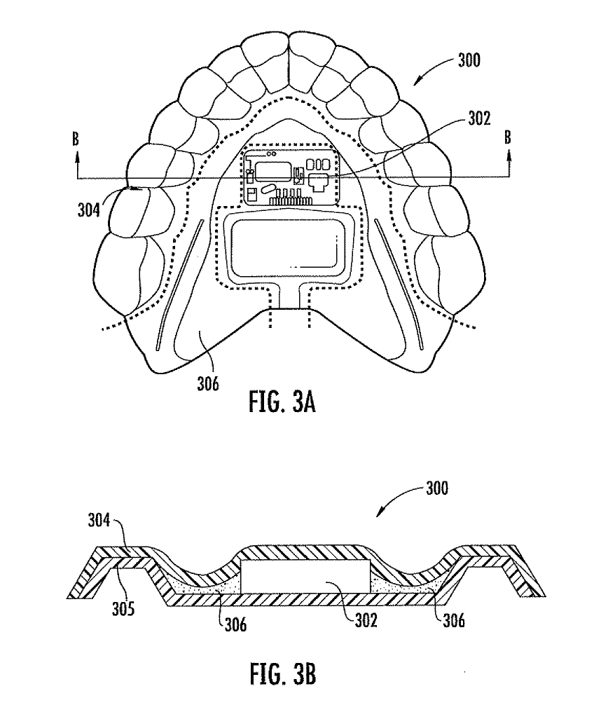

An example of the intra-oral appliance or mouthpiece 300 is illustrated in FIGS. 3A and 3B. The electronics module 302 is sandwiched between upper and lower protective layers 304, 305 (e.g. such as thermoformed plastic layers) for protection of the circuitry from saliva and associated corrosion. Also, an adhesive layer 306 (e.g. a bonded, light-cured, acrylic gel, such as Triad Gel from the Dentsply International of York, Pa.) is preferably applied between the protective layers, e.g. at a periphery thereof, to further aid in the corrosion prevention.

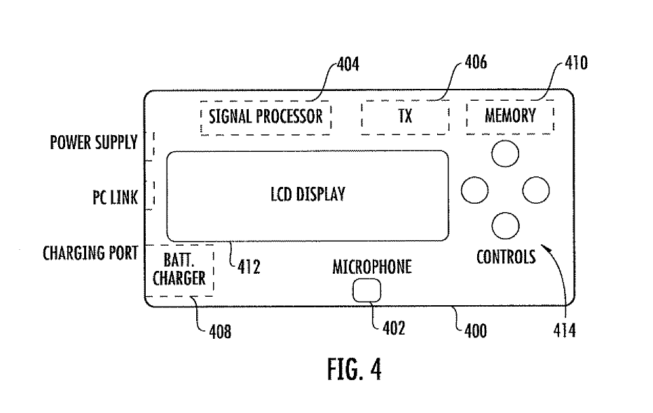

FIG. 4 is an illustration of the extra-oral electronic transceiving (nightstand) unit 400 which may be located on the patient's nightstand. It contains the microphone 402, a signal processor 404, and a wireless transceiver 406 to activate the intra-oral appliance. It also includes a battery charger 408 for the appliance and a data recorder 410 to monitor snoring/gasping frequency throughout the night. The location of the LCD display 412, microphone 402 and the controls 414 may be located as desired anywhere on the housing of the nightstand unit 400. In use, these components can be located not only on a nightstand but also anywhere proximate to the patient that may be desired.

The battery charger 408 of the extra-oral unit 400 and the associated battery 302 of the intra-oral unit may utilize connectors manufactured by 3M such as 0.100″ pin strip headers and 0.100″ board mount sockets. The socket is used in the mouthpiece and is sealed within the protective thin plastic layers by applying bonded, light-cured, acrylic gel, such as Triad Gel from the Dentsply International of York, Pa., to prevent moisture from entering the mouthpiece. Contactless charging, such as electromagnetic, capacitive and/or inductive charging may also be provided instead of the connectors.

To detect a snoring pattern, a computing element such as a microcontroller, monitors incoming audio signals from the microphone. When this becomes greater than or equal to the user-set threshold, electrical stimulation occurs. The active low pass filter attenuates sounds greater than 1 khz. Previous studies have identified a narrow band in which the majority of snoring sounds occur and with selective amplification of the input, bed partner and background noise will not reach threshold. The microphone input is relative to the distance from the noise source. Distance from the microphone on the nightstand next to the snorer and adjustability of sensitivity will prevent accidental activation.

Snoring does occur in variable patterns that will be recorded relative to timing and amplitude. The LCD screen shown in FIG. 4 has a line for displaying the number of snores over an eight-hour period and downloading the stored data to a computer program will produce a graph showing when the snores occurred, the number, and loudness. The patient will be able to evaluate their snoring with the intra-oral appliance in or out of the mouth and can set a time delay upon retiring before activation of the appliance. Evaluation of the recorded data will guide the adjustments to maximize the benefit for individual differences.

A PC link allows data transfer for home computer analysis and tracking of abnormal breathing sounds with and without the appliance in place. This will give the patient feedback on breathing difficulties during sleep and benefit of the appliance. The device functions by the extra-oral electronic unit detecting snoring sounds and, consequently, transmitting a wireless signal to the intra-oral appliance which, in turn, generates a low voltage current which is carried to the patient's hamular notches causing the soft palate to flex or stiffen aiding in the opening of the airway and restoring air flow to the patient's lungs.

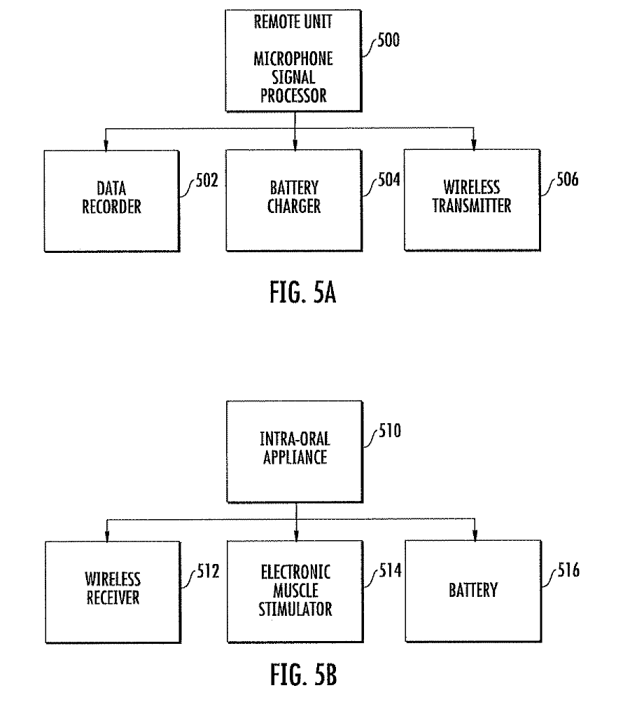

FIGS. 5A and 5B show a simplified chart of the electronic functions of a first version of the remote unit and intra-oral appliance, respectively. More specifically, referring to FIG. 5A, the extra-oral unit or remote unit operations include the microphone function and signal processing 500 which are associated with data recording 502, battery charging 504 and wireless transmissions 506. Referring now to FIG. 5B, the intra-oral appliance operations 510 are associated with wireless reception 512, electronic muscle stimulation 514 and power supply 516 from the battery.

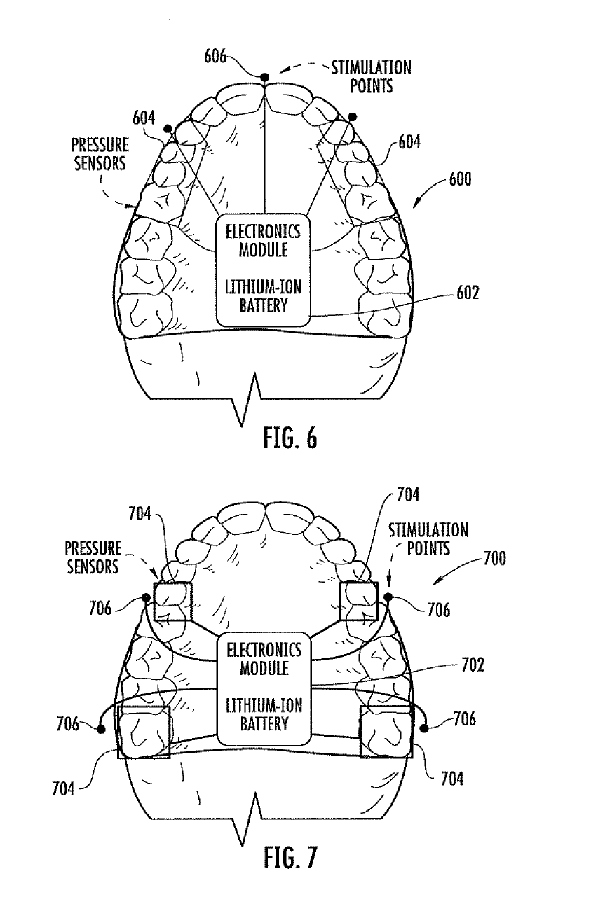

FIG. 6 is an illustration of the intra-oral appliance 600 for bruxism. This electronic orthosis works as a gnathologic appliance to protect teeth from damage during excursive movements. In addition, the electronics package 602 detects bruxing activity using a pressure electro conductive rubber sensor or pressure receptor switch 604 such as made by Bridgestone in Tokyo, Japan and stops it with electronic stimulation, via tissue contact 606, to the intra-oral mucosa at a subconscious level without sleep interruption. Patient adjustability and monitoring is available with the extra-oral unit, discussed above, that is in wireless communication with the intra-oral appliance.

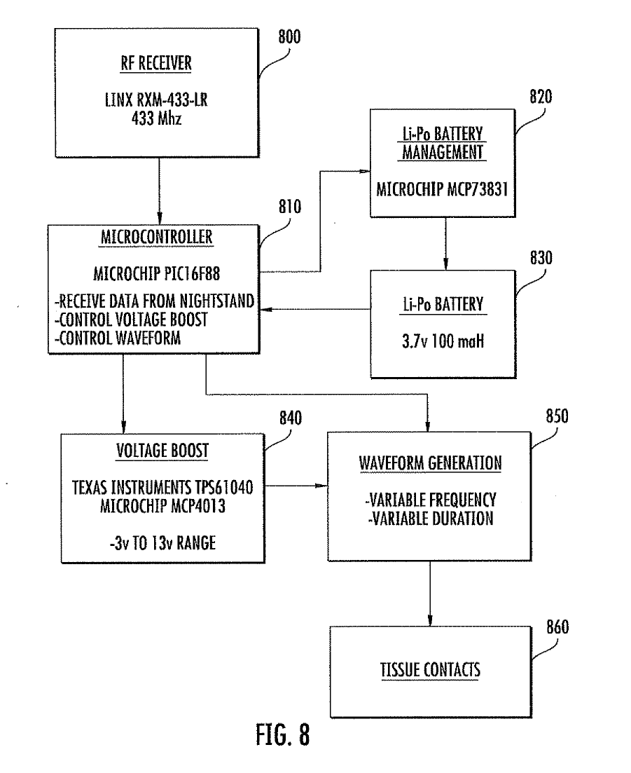

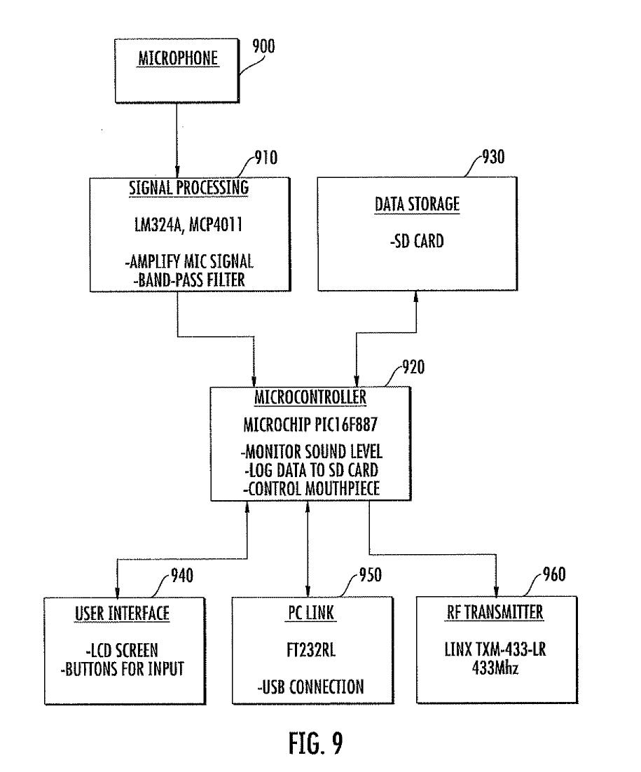

FIG. 7 is an illustration of the intra-oral appliance 700 for TMD. Temporomandibular disorder (TMD), or TMJ syndrome, is a term covering acute or chronic inflammation of the temporomandibular joint, which connects the lower jaw to the skull. This orthotic type appliance detects oral para-functional activity through the use of pressure sensors 704 and an electronics package 702 in the appliance. A para-functional habit or parafunctional habit is the habitual exercise of a body part in a way that is other than the most common use of that body part. The term is most commonly used by dentists, orthodontists, or maxillofacial specialists to refer to parafunctional uses of the mouth, tongue and jaw. Oral para-functional habits may include bruxism (tooth-clenching or grinding), tongue tension, mouth-breathing, and any other habitual use of the mouth unrelated to eating, drinking, or speaking. Treatment includes electronic stimulation, via tissue contact 706 in response to detected pressure.

Wireless communication with the extra-oral unit provides data storage and patient adjustability for electrical stimulation in voltage, frequency, pulse width, and duration.

FIG. 8 is a high-level block diagram of a preferred hardware architecture of the mouthpiece unit in accordance with one aspect of the invention. An RF receiver 800, such as receiver RXM-433-LR manufactured by Linx Technologies, Inc. of Merlin, Oreg., receives signals transmitted by the nightstand unit, described hereinafter. Signals from the RF receiver are passed to a computing element such as controller or microcontroller 810 which is preferably a PIC16F88 microcontroller manufactured by Microchip Technology Inc. of Chandler, Ariz. The Voltage Boost 840 receives the output of the RF receiver and provides a voltage boost, preferably using switch boost converter TPS61040 manufactured by Texas Instruments, Inc. of Dallas, Tex., to boost the voltage as specified by the microcontroller. The microcontroller also controls the shape of the waveform generator 850 to vary the frequency and duration of the waveforms applied to the mouth of the patient through tissue contacts 860. Control of the intensity of the waveform can be exerted using an MCP4013 Digital Potentiometer manufactured by Microchip Technology, Inc. of Chandler, Ariz.

FIG. 9 is a high-level block diagram of a preferred hardware architecture of the nightstand unit in accordance with one aspect of the invention. The so-called nightstand unit includes a microphone 900, the purpose of which is to detect sounds that occur during sleep. For purposes of this application it is called a nightstand unit although the particular unit or its components can be located anywhere in the vicinity of the person who might be the subject of a sleep-disordered breathing. Sounds picked up by the microphone 900 during operation of the unit, usually at night, is passed to a signal processing unit 910. The purpose of the signal processing unit 910 is to amplify the signal from the microphone and shifts it into the 0 to 5 volt range, preferably. This is preferably done using a quad operational amplifier LM324A manufactured by STMicroelectronics of Phoenix, Ariz. The processed signal from 910 is passed to a computing element such as the controller or microcontroller 920 which is preferably a microcontroller PIC16F887 manufactured by Microchip Technology, Inc. of Chandler, Ariz. The amplified signal is sampled by microcontroller 920 and the sample stored in a data storage unit 930 which is preferably a standard SD memory card where it will be stored. The microcontroller 920 is programmed, as described more hereinafter and in the source code CD provided with this application, to monitor the sound level in the room. When the level indicates that a certain sleep-breathing disorder is present, such as snoring, it sends a signal to the RF transmitter 960 to activate the mouthpiece unit, previously described. This results in electrical stimulation of the oral cavity of the patient at the tissue contacts 860 shown in FIG. 8. The electrical stimulation is set so as not to interrupt the sleep of the patient but rather to stimulate the oral cavity to aid in opening the patient's partially or totally collapsed airway. The nightstand unit also includes a link to a personal computer 950 which may be either a wired connection or a wireless connection over which data from the data storage unit 930 can be downloaded and analyzed. Access to the microcontroller is also provided over user interface 940 which displays information from the microcontroller and allows the user to activate buttons or controls to indicator set various preferences with respect to the operation of the unit.

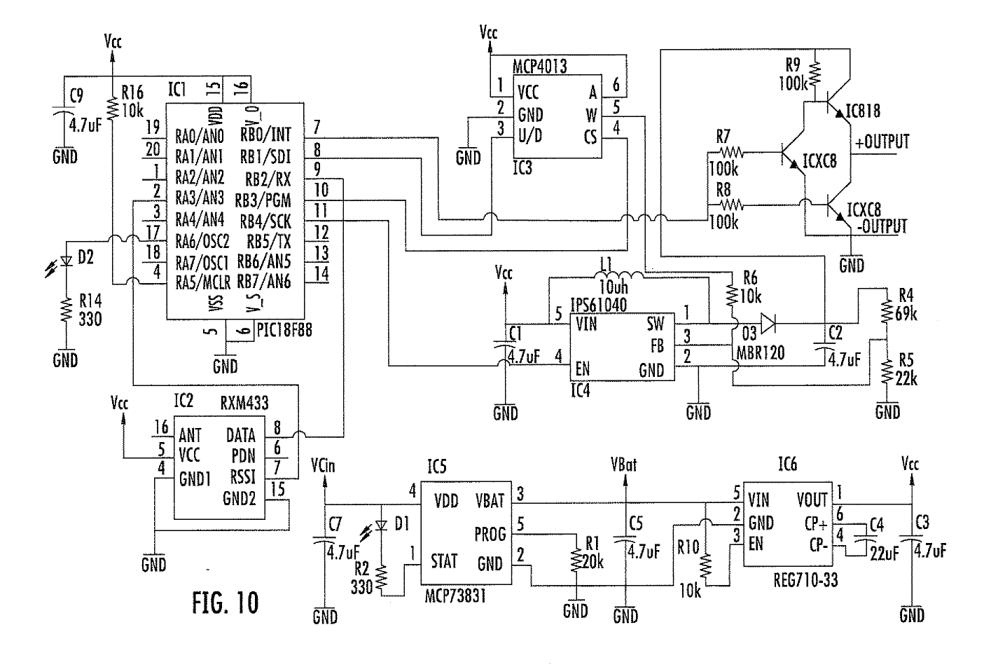

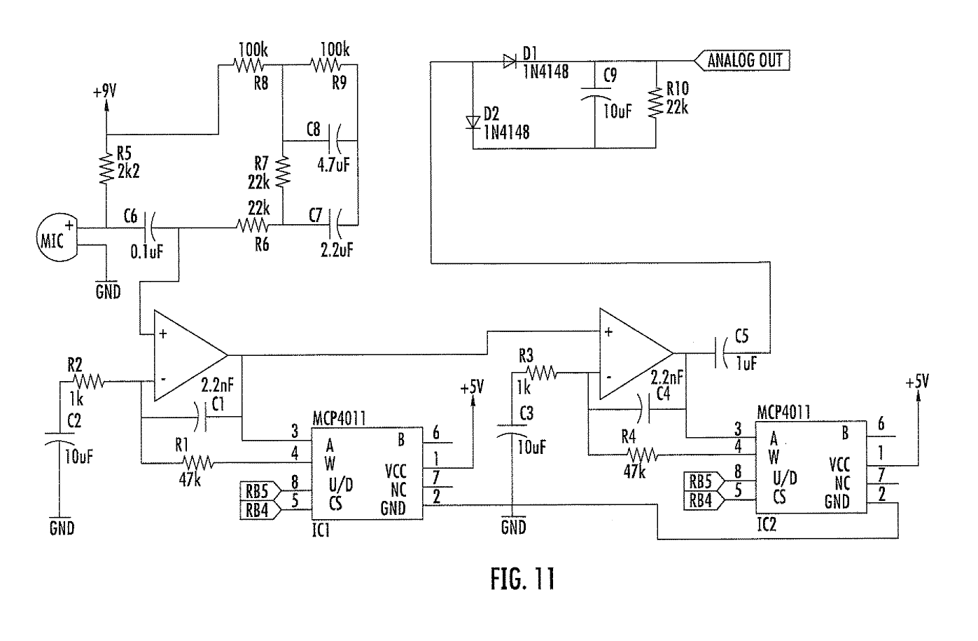

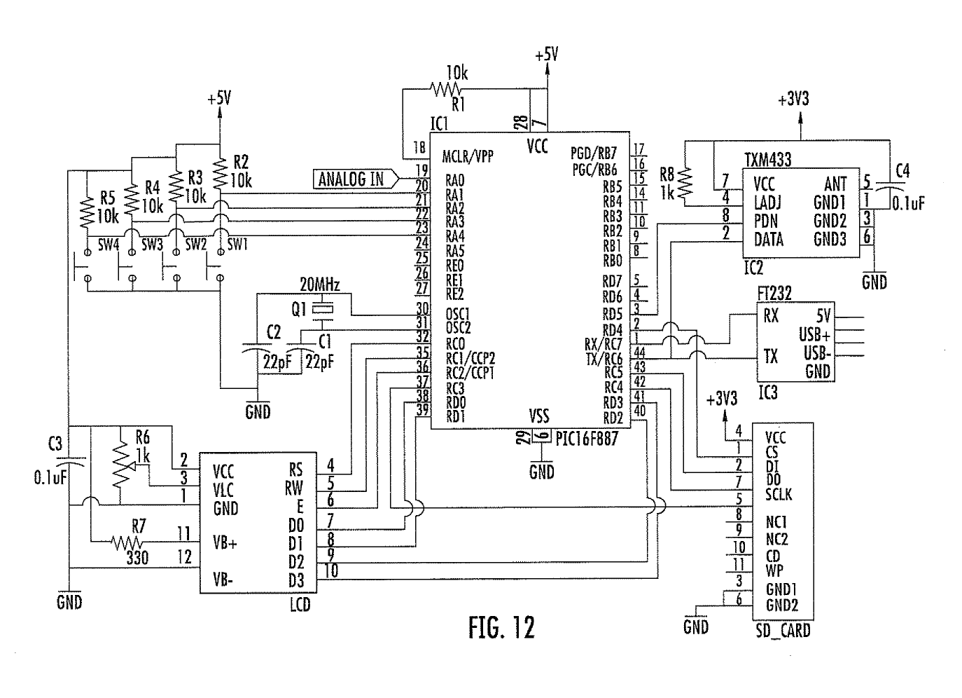

Referring to FIGS. 10-12, more detailed schematic diagrams of an embodiment of the mouthpiece unit and nightstand unit are illustrated. More specifically, FIG. 10 illustrates the various integrated circuit chips and connections of an embodiment of the mouthpiece of FIG. 8 including the microcontroller, RF receiver, battery and associated battery management, voltage boost, waveform generation and tissue contacts as shown. FIG. 11 illustrates the various integrated circuit chips and connections of an embodiment of the microphone and signal processing circuitry of the nightstand unit of FIG. 9. FIG. 12 illustrates the various integrated circuit chips and connections of an embodiment of the microcontroller, data storage, user interface, PC link and RF transmitter of the nightstand unit of FIG. 9.

Exchange of information between the mouthpiece and the nightstand unit occurs in data packets. A single (nightstand) unit can service up to 256 mouthpiece units on separate channels.

The mouthpiece knows what channel it is on and will not respond to any data packets that are not addressed to its specific channel.

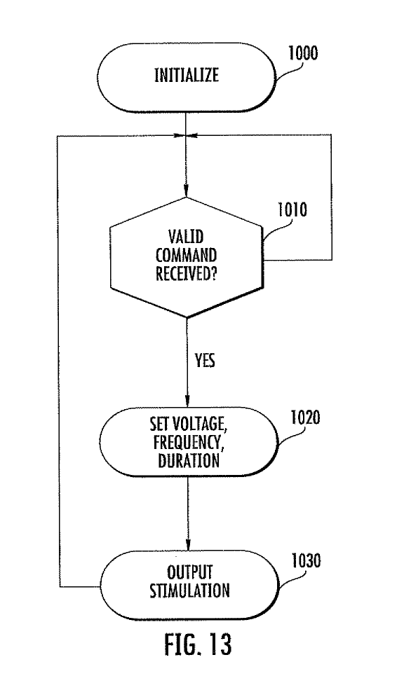

FIG. 13 is a high-level block diagram of the software architecture implemented in firmware for the mouthpiece unit in accordance with one aspect of the invention.

At a high-level, the firmware for the mouthpiece has an initialization state 1000 which readies the mouthpiece unit to receive signals from the nightstand unit. If the mouthpiece unit receives a valid command or signal from the nightstand unit (1010) the voltage, frequency and duration is set (1020) and the output stimulation, corresponding to the setting, is applied to the patients oral cavity. Once stimulated, the mouthpiece software waits until another command is received. This process loops throughout the night, until the device is turned off when the patient awakes in the morning.

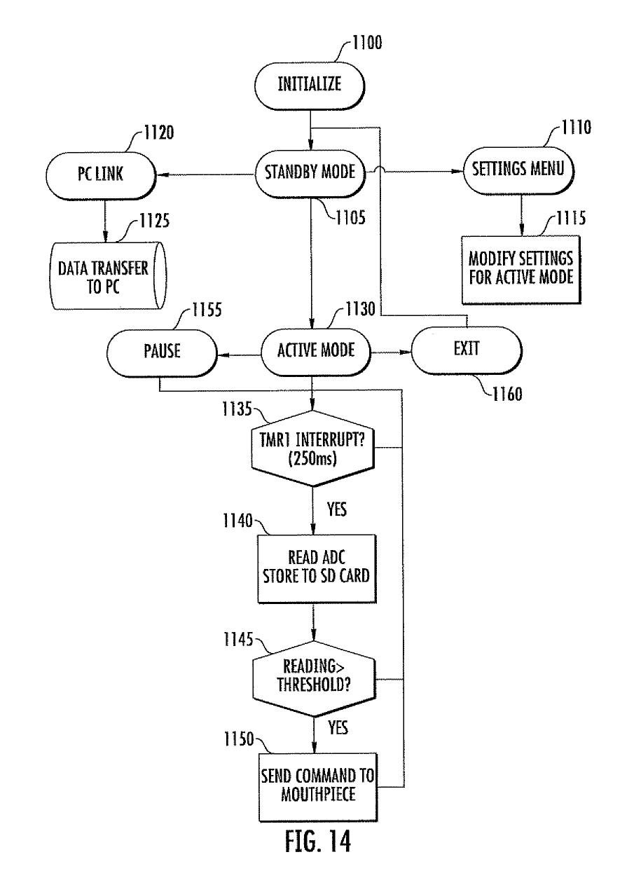

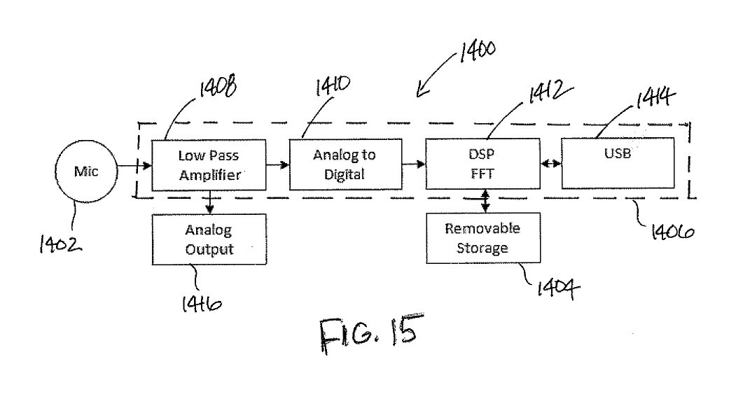

FIG. 14 is a high-level block diagram of the software architecture implemented in firmware for the nightstand unit in accordance with one aspect of the invention.

When turned on, the nightstand software initializes (1100) the nightstand unit for operation.

The software then enters a standby mode 1105. In the standby mode, the nightstand unit can receive settings set by a user through a settings menu 1110. The settings also permit the software to be transitioned into active mode (1115). From the standby mode 1105, the software can also enter into a linking operation with a computing device, such as a personal computer over PC link 1120. When in communication with the PC over PC link 1120, the nightstand unit can transfer data to a computing device where it can be stored and analyzed (1125). In active mode, the software enters a sampling loop during which the level of signal received from the microphone is sampled by asserting a timed interrupt, preferably every 250 milliseconds. The sampled signal will be converted to digital using an analog-to-digital function and the results stored in a storage unit, such as an SD card (1140) for later analysis. If the sampled value is above a threshold (1145) a command is sent to the mouthpiece (1150) where it is received and, as previously discussed, will be utilized to initiate electrical stimulation of the patient's oral cavity. This timer driven interrupt sequence occurs repeatedly throughout the night but may be paused (1155) or exited (1160) upon user action.

This new appliance detects and records specific snoring frequencies with a nightstand unit that selectively activates a wireless gnathodynamics based electronic intra-oral appliance to stop the snore. A low voltage electrical stimulation of the levator and tensor palatine muscles stops the snore. The resulting increase in muscle tonicity restores the airway and prevents vibration of the soft palate without awaking the patient. It is prescribed by the dentist and fabricated by a certified dental laboratory using pre-packaged electronic circuitry and a rechargeable battery that is encapsulated between two layers of thermoformed material. The mandible is positioned anatomically considering the temporomandibular joints, muscles, and teeth. All teeth are in contact to prevent extrusion and all eccentric movements are sheltered with a mutually protected occlusal scheme built into the appliance with no anterior repositioning or excessive mandibular opening. Overnight data is recorded preferably every 250 msec and stored for download to any PC. Analysis of stored data by the dentist preferably guides adjustments for muscle stimulation relative to intensity, duration, frequency, sensitivity, and time delay.

Electronic muscle stimulation restores tone while sleeping to that experienced during the day. The increased tonicity prevents the soft palate from vibrating on inspiration and expiration. In initial clinical trials to determine that the invention works, the results with four chronic snoring patients showed effectiveness, patient acceptance, and ease of use, have been exceptional. A statistically significant decrease in snoring sound levels were recorded. Witnesses confirmed decreased snoring activity and patients stated that they felt more rested and were having dreams (REM sleep) again. Pulse oximetry data shows increased average oxygen saturation levels with appliance use. No occlusal changes, patient discomfort, or TMD symptoms were noted after four months of wear.

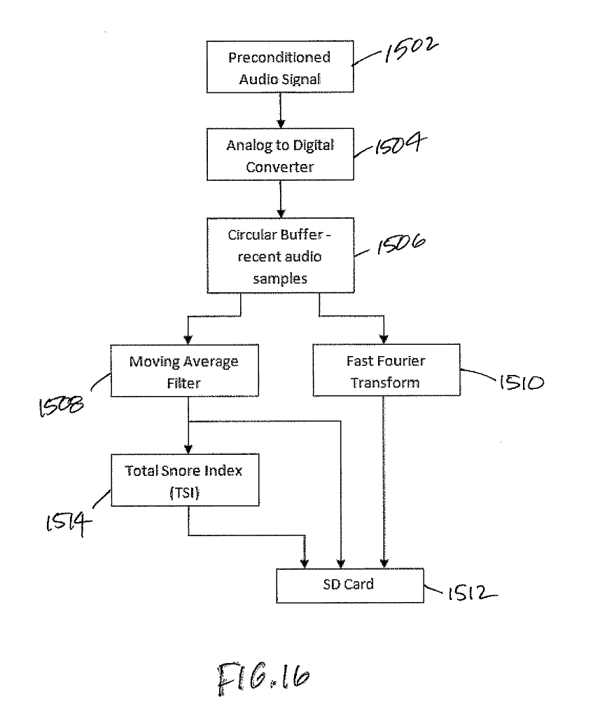

Referring now to FIGS. 15 and 16, an additional embodiment of the extra-oral unit being used as a snore recorder will now be described. This functional use or mode of operation may be referred to as the SRD (Snore Recording Device), which involves using the extra-oral unit as a snore recorder and data storage device 1400 for sleep lab studies and/or take home overnight use. In this embodiment, the extra-oral unit 400 (FIG. 4) does not initiate patient stimulations and does not communicate with the oral appliance 300. The snore recording can be pre/post treatment and used as a baseline screening for snoring or a follow-up to evaluate treatment. The SRD 1400 interfaces directly with the recorded data during an in-lab PSG or can be used as a multiple night take home test. The recorded snore data may produce a report which shows total number of snores, time domain, frequency domain, and a Total Snore Index (TSI) score for each sleep period.

The SRD 1400 is an electronic, microcontroller based, device that has two primary functions. First, it interfaces with the polysomnograph equipment to record and input a high quality accurate graph and analysis of breathing sounds during a PSG in a sleep lab. This additional data will enhance the diagnostic capability of the PSG and provide valuable information to pre and post treatment evaluations.

Secondly, the SRD 1400 can be sent home with the patient for multiple nights recording of sleep breathing sounds to screen for sleep disordered breathing or as a follow-up evaluation of effectiveness of oral appliance therapy used for snoring and mild to moderate sleep apnea prior to a formal in-lab sleep study. Effectiveness of snoring and OSA treatments, such as oral appliances, can be better evaluated both in the sleep lab, and at home, with the portable SRD 1400.

Frequency, timing, amplitude, and decibel levels will be accurately recorded and provided to the PSG for analysis and evaluation in diagnosis and treatment of sleep disordered breathing. The Total Snore Index (TSI) score calculated from the collected snore data may be standardized for both longitudinal and cross-sectional comparison.

The SRD 1400 can also be used as a take home monitor with multiple overnight sound data stored on a memory card that can be downloaded to a PC for review and analysis. The SRD 1400 is preferably battery powered. It can be utilized as a screening tool following a positive history of sleep disordered breathing or a score above 9, for example, on the Epworth Sleepiness Scale. The take home use will also be valuable in confirming effectiveness of treatment for snoring without the cost and inconvenience of an in-lab sleep study.

The snore recording device 1400 includes a portable housing (FIG. 4), a microphone 1402 carried by the housing for capturing an audio input signal including snoring, a memory 1404, such as a removable memory, carried by the housing, and processing circuitry 1406 carried by the housing and coupled to the microphone and the memory. The processing circuitry 1406 is for low pass filtering, e.g. via low pass filter/amplifier 1408, the audio input signal from the microphone 1402 to generate a low pass filtered analog signal. The processing circuitry 1406 performs analog-to-digital conversion (ADC), e.g. via A/D converter 1410, on the low pass filtered analog signal to generate an intermediate digital signal. The processing circuitry 1406 performs a moving average filtering, e.g. via DSP (FFT) 1412, of the intermediate digital signal to generate moving average intensity data, and performs a Fast Fourier Transform (FFT) on the intermediate digital signal to generate frequency component data. The moving average intensity data and frequency component data may be stored in the memory 1404, such as a removable memory card.

The processing circuitry may also be for calculating, from the moving average intensity data, snoring index data (e.g. TSI 1514) based upon a number of snoring events per unit time, and storing the snoring index data in the memory 1404. The processing circuitry may also be for amplifying 1408 the audio input signal from the microphone 1402. The processing circuitry may also be for storing the low pass filtered analog signal in the memory. The processing circuitry may further comprise a polysomnograph (PSG) interface (e.g. USB 1414) for interfacing to a PSG. The processing circuitry 1406 may also be for performing a circular buffering of the intermediate digital signal.

The main function of the SRD 1400 firmware (FIG. 16) is to acquire and analyze sound data. The audio signal is captured by an electret-microphone 1402 and then passed through several low-pass filters and gain stages to precondition 1502 the signal for the analog to digital converter 1504. Frequencies higher than 500 Hz may be attenuated as they are higher than typical snoring sounds. The preconditioned audio signal may also be output 1416 for direct integration into a typical sleep lab's polysomnography (PSG) equipment.

After the audio signal has been digitized, sound samples are stored in a circular buffer 1506 for increased analytical efficiency. The moving average 1508 is calculated to provide the intensity level of the audio signal. The Total Snore Index (TSI) 1514 is calculated based on number of snoring events detected from the output of the moving average filter. The TSI represents the number of snores per hour of sleep.

The Fast Fourier Transform 1510 is calculated from the audio sample data in the circular buffer. This data provides the frequency content of the audio signal. The frequency content has been shown to differ between patients with normal snoring and obstructive sleep apnea (OSA). The audio signal intensity and frequency content are continuously stored to a form of removable media 1512, typically a SD card. This data can then be transferred to a computer via USB 1414 for graphical analysis.

While various embodiments of the present invention have been illustrated herein in detail, it should be apparent that modifications and adaptations to those embodiments may occur to those skilled in the art without departing from the scope of the present invention as set forth in the following claims.

What is claimed is:

1. A snore recording device comprising:
a portable housing;
a microphone carried by said housing for capturing an audio input signal including snoring;
a memory carried by said housing;
processing circuitry carried by said housing and coupled to said microphone and said memory for
low pass filtering the audio input signal from said microphone to generate a low pass filtered analog signal,
performing analog-to-digital conversion (ADC) on the low pass filtered analog signal to generate an intermediate digital signal,
performing a moving average filtering of the intermediate digital signal to generate moving average intensity data,
performing a Fast Fourier Transform (FFT) on the intermediate digital signal to generate frequency component data, and
storing at least the moving average intensity data and frequency component data in said memory.

2. The snore recording device of claim 1 wherein said processing circuitry is also for calculating, from the moving average intensity data, snoring index data based upon a number of snoring events per unit time, and storing the snoring index data in said memory.

3. The snore recording device of claim 1 wherein said processing circuitry is also for amplifying the audio input signal from said microphone.

4. The snore recording device of claim 1 wherein said processing circuitry is also for storing the low pass filtered analog signal in said memory.

5. The snore recording device of claim 1 wherein said processing circuitry further comprises a polysomnograph (PSG) interface for interfacing to a PSG.

6. The snore recording device of claim 1 wherein said processing circuitry is also for performing a circular buffering of the intermediate digital signal.

7. A snore recording device comprising:
a portable housing;
a microphone carried by said housing for capturing an audio input signal including snoring;
processing circuitry carried by said housing and coupled to said microphone and said memory for
low pass filtering the audio input signal from said microphone to generate a low pass filtered analog signal,
performing analog-to-digital conversion (ADC) on the low pass filtered analog signal to generate an intermediate digital signal,
performing a moving average filtering of the intermediate digital signal to generate moving average intensity data,
performing a frequency domain analysis on the intermediate digital signal to generate frequency component data,
calculating, from the moving average intensity data, snoring index data based upon a number of snoring events per unit time, and
outputting at least the moving average intensity data, snoring index data and frequency component data to a removable memory.

8. The snore recording device of claim 7 wherein said processing circuitry is also for amplifying the audio input signal from said microphone.

9. The snore recording device of claim 7 wherein said processing circuitry is also for outputting the low pass filtered analog signal to the removable memory.

10. The snore recording device of claim 7 wherein said processing circuitry further comprises a polysomnograph (PSG) interface for interfacing to a PSG.

11. The snore recording device of claim 1 wherein said processing circuitry is also for performing a circular buffering of the intermediate digital signal.

12. A method for recording snores comprising:
capturing an audio input signal including snoring;
low pass filtering the audio input signal to generate a low pass filtered analog signal;
performing analog-to-digital conversion (ADC) on the low pass filtered analog signal to generate an intermediate digital signal;
performing a moving average filtering of the intermediate digital signal to generate moving average intensity data;
performing a Fast Fourier Transform (FFT) on the intermediate digital signal to generate frequency component data; and
storing at least the moving average intensity data and frequency component data in a memory.

13. The method of claim 12 further comprising calculating, from the moving average intensity data, snoring index data based upon a number of snoring events per unit time, and storing the snoring index data in the memory.

14. The method of claim 12 further comprising amplifying the audio input signal.

15. The method of claim 12 further comprising storing the low pass filtered analog signal in the memory.

16. The method of claim 12 further comprising providing at least the moving average intensity data and frequency component data to a polysomnograph (PSG).

17. The method of claim 12 further comprising performing a circular buffering of the intermediate digital signal.
